(19) United States
(12) Patent Application Publication
(10) Pub. No.: US 2022/0111161 A1
(43) Pub. Date: Apr. 14, 2022

(54) Vaporization Apparatus and Authorization System

(71) Applicant: Holding Dragons, LLC, Salt Lake City, UT (US)

(72) Inventors: Matthew Hawkes, Herriman, UT (US); Mai Nguyen, Salt Lake City, UT (US); Sean Dang, Salt Lake City, UT (US)

(21) Appl. No.: 17/341,133

(22) Filed: Jun. 7, 2021

Related U.S. Application Data
(60) Provisional application No. 63/090,697, filed on Oct. 12, 2020.

(51) Int. Cl. A61M 15/06; G05B 19/042; A24F 40/53; A24F 40/60; A24F 40/57; A24F 40/65; A61M 11/04

(52) U.S. Cl. CPC A61M 15/06; G05B 19/0426; A24F 40/53; A24F 40/60; A24F 40/57; A61M 2205/52; A61M 11/042; G05B 2219/24167; A61M 2205/6009; A61M 2205/502; A61M 2205/8206; A24F 40/65

(57) ABSTRACT

A battery for a vaporization apparatus includes a data storage device configured to read authorization data for an authorized user of the vaporization apparatus and a user interface configured to receive a security input from a user. The battery also includes an electronic controller configured to communicate electronically with the user interface and the data storage device, wherein the electronic controller is further configured to receive the security input from the user interface, compare the received security input with the authorization data to identify the authorized user in an authentication process, and authorize use of the vaporization apparatus.

### RELATED APPLICATIONS

This application claims the benefit of U.S. Provisional Patent Application No. 63/090,697, filed Oct. 12, 2020, which is incorporated herein by reference in its entirety.

### BACKGROUND

Identifying and verifying the proper user for a vaporization apparatus is important for administering proper dosages of medication to the user and preventing overuse of the apparatus. Ensuring the correct user is using the vaporization apparatus is also important for verifying that the proper type of medication is being administered at the right frequency, beyond ensuring the right dose is being administered.

### SUMMARY

Various embodiments are described herein to facilitate coordination and control of the administration of medical or other substances to users under the guidance of a professional administrator such as a doctor. When a prescription is provided to a patient, for example, embodiments described herein may help ensure that the patient follows recommended dosages and dosage timelines.

Different types of authorization controls are described with reference to embodiments herein. In one embodiment, biometric security is implemented to confirm that a user is, in fact, the correct patient for the medication requested. In another embodiment, a touch screen or other user input device is used to acquire user-specific biometric information for the biometric security confirmation. In another embodiment, the actual container or dispenser holding the medication or other substance is “smart” by the integration of electronics that can control when the medication can be dispensed to a specific user. In further embodiments, location tracking and finding functionality is integrated into the medication dispenser so that a user can locate otherwise lost medications.

These types of functionality, and other functionality, may improve the security and accessibility of proper medications and dosages to users in need of those medications and other substances. While many benefits are perceived and potentially available through embodiments described herein, some of the embodiments facilitate specific benefits. For example, in one embodiment, user authentication for the use of a specific pod of medication can allow a user to swap batteries while still authenticating the necessary user information with a back-end data system. The use of a backend system also may facilitate over air updates of hardware or software within the device. These and various other benefits may be achieved by embodiments described herein.

### BRIEF DESCRIPTION OF THE DRAWINGS

FIG. 1 depicts a schematic diagram of one embodiment of a vaporization apparatus.

FIG. 2 depicts a schematic diagram of one embodiment of a system for dispenser authorization controls.

FIG. 3 depicts a schematic diagram of one embodiment of the battery of FIG. 1.

FIG. 4 depicts a schematic diagram of one embodiment of the pod of FIG. 1.

FIG. 5 depicts a flow chart diagram of one embodiment of a method for authenticating user requests.

FIG. 6 depicts a schematic perspective diagram of one embodiment of a vape device.

FIG. 7 depicts a plan view of the vape device of FIG. 6.

FIG. 8 depicts an exploded view of the vape device of FIG. 6.

FIG. 9 depicts one embodiment of an end view of the vape device of FIG. 6.

FIG. 10 depicts one embodiment of various interface messages of the vape device of FIG. 6.

FIG. 11 depicts embodiments of interface messages of the vape device of FIG. 6.

Throughout the description, similar reference numbers may be used to identify similar elements.

### DETAILED DESCRIPTION

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but it is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Reference to a computer readable medium may take any physical form capable of storing machine-readable instructions, at least for a time in a non-transient state, on a digital processing apparatus. A computer readable medium may be embodied by a compact disk, digital-video disk, a Blu-ray disc, a magnetic tape, a Bernoulli drive, a magnetic disk, flash memory, integrated circuits, or other digital processing apparatus memory device.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment,” “an embodiment,” or similar language means that a particular feature, structure, or characteristic described in connection with the indicated embodiment is included in at least one embodiment of the present invention. Thus, the phrases “in one embodiment,” “in an embodiment,” and similar language throughout this specification may, but do not necessarily, all refer to the same embodiment.

While many embodiments are described herein, at least some of the described embodiments facilitate authorization of user access to different versions of medical or other substance dispensers. The use of authorization and access controls to different dispensers allows administrators or medical professionals to be involved in and have oversight of the use of the substance(s) prescribed to or otherwise made available to the users. Embodiments of this type of authorization and user access control may be beneficial to help mitigate the potential misuse of such products by unauthorized users Similarly, embodiments of this type of authorization of user access control may be beneficial to help mitigate the potential misuse of such products by the authorized users themselves. For example, in some embodiments an authorized user maybe restricted from inadvertently or purposefully dispensing a dose that is different from what has been prescribed or authorized.

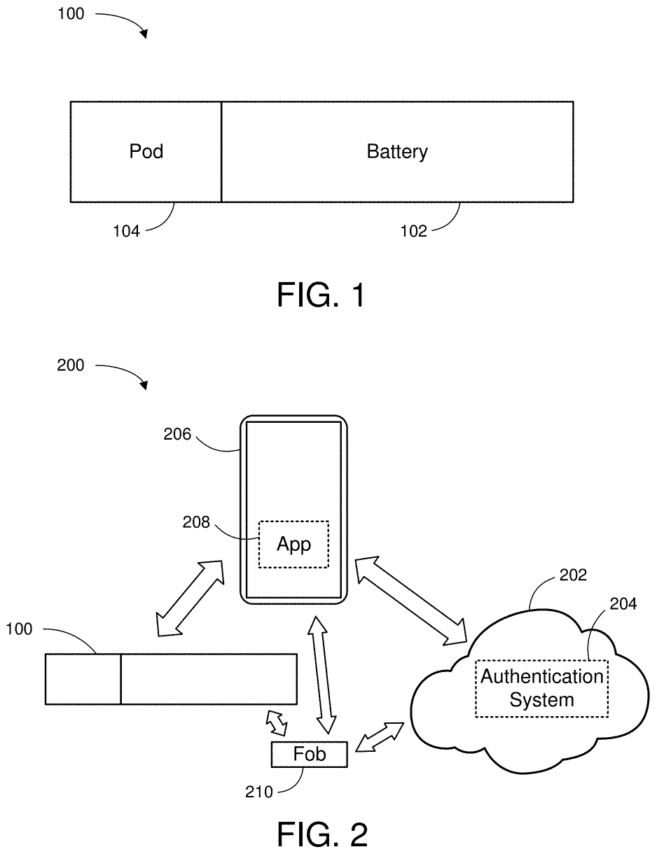

FIG. 1 depicts a schematic diagram of one embodiment of a vaporization apparatus 100. The illustrated vaporization apparatus 100 includes a battery 102 and a pod 104. Although the vaporization apparatus 100 is shown and described with certain components and functionality, other embodiments of the vaporization apparatus 100 may include fewer or more components to implement similar or different functionality.

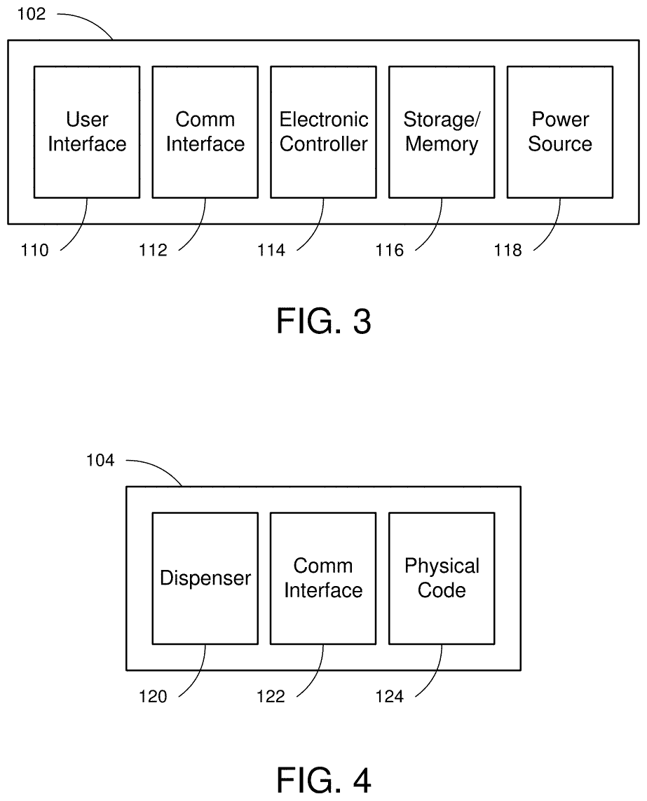

In general, the battery 102 provides power and functionality to the overall apparatus 100. In some embodiments, the battery 102 provides a power source and connectivity to the pod 104. This allows the pod 104 to be powered for dispensing medicine or another substance, including a vaporized gas, to a user. One embodiment of the battery 102 is shown in more detail and described with reference to FIG. 3.

The pod 104 connects into the battery 102 to receive power from the battery 102. The pod 104 also includes a repository for medication or another substance to be stored within the pod 104. When activated, the battery 102 provides power to the pod 104 to heat up and convert the substance in the pod 104 into a vapor or gas. A more detailed embodiment of the pod 104 is shown and described with reference to FIG. 4.

FIG. 2 depicts a schematic diagram of one embodiment of a system 200 for dispenser authorization controls. In the illustrated embodiment, the system 200 includes the vaporization apparatus 100 connected to, or in communication with, a variety of other components.

In one embodiment, the system 200 includes communications connectivity, either directly or indirectly, to the cloud 202 or another centralized data storage location. An authentication system 204 is available to help provide controls and policies for authorized use of the vaporization apparatus 100. The authentication system 204 may include various types of data storage and processing capabilities. For example, in some embodiments, the authentication system 204 includes a data registry for various users to store data about each user and the types of medications they are able to access, including the types of dosages and timing of such access.

In some embodiments, authorized administrators such as doctors are able to upload user profiles to the data registry. Authorized administrators are also able to upload medication and dosage information for each user. In some embodiments, authorized administrators are also able to view usage statistics or data for those users in order to review the types of medications and dosages that have been accessed, or that the user has tried to access.

The system 200 also includes a portable user device such as a phone 206 or tablet. Although a phone 206 is referenced throughout this description, other embodiments of computing devices may be implemented and are within the scope of embodiments herein. In an embodiment, the phone 206 includes a software application 208 that is configured to facilitate certain communications with the authentication system 204 and the vaporization apparatus 100. The software application or app 208 helps facilitate communications of user data and/or authorizations for proper access control to the vaporization apparatus 100 by a user.

The illustrated system 200 also includes a fob 210 that can communicate with any or all the other components of the system 200. In some embodiments, the fob 210 can be programmed with data from the authentication system 204 and/or the app 208. In this way, the fob 210 can be used as a setup device to communicate with the vaporization apparatus 100 to initially load user data, medication data, or other data from the fob 210 to a storage or memory device within the vaporization apparatus 100 (either on the battery 102 or within the pod 104).

FIG. 3 depicts a schematic diagram of one embodiment of the battery 102 of FIG. 1. The illustrated battery 102 includes several components for the operation of the overall vaporization apparatus 100. In one embodiment, the battery 102 includes a user interface 110, a communication interface 112, an electronic controller 114, a storage or memory device 116, and a power source 118. Although the battery 102 is shown and described with certain components and functionality, other embodiments of the battery 102 may include fewer or more components to implement similar or different functionality.

In one embodiment, the user interface 110 allows the user of the vaporization apparatus 100 to input commands to the vaporization apparatus 100 and/or to receive data notifications from the vaporization apparatus 100. The specific type of user interface 110 that is implemented may depend on the types of user commands and controls that are processed by the electronic controller 114. In some embodiments, the user interface 110 is a touch interface. In some embodiments, the user interface 110 is a LED or LCD screen. In other embodiments, the user interface 110 includes any other type of interface which allows touch, voice, or other user controls to be input and processed by the electronic controller 114.

In one embodiment, the communication interface 112 facilitates electronic or digital communications with other components within the vaporization apparatus 100 and the system 200. The communication interface 112 may include physical and wireless types of communication between hardware and software. For example, in some embodiments the communication interface 112 includes contact terminals to directly interface with corresponding contact terminals on the pod 104. In other embodiments, the communication interface 112 includes hardware and controllers to facilitate communications with the pod 104, the app 208, the fob 210, or other components of the system 200 using Wi-Fi, Bluetooth, near field communication (NFC), or other communication protocols.

In one embodiment, the electronic controller 114 includes all hardware and software in any format necessary or beneficial to the functionality of the battery 102 and the various functions described herein. The electronic controller 114 may be implemented in a printed circuit board (PCB), programmable chip, or any other electronics-based device capable of implementing logic and/or processing applicable software commands. For example, the electronic controller 114 may be configured, in some embodiments, to process input from the user interface 110 to detect a user fingerprint or recognize a user voice as part of the user authentication process. In other embodiments, the electronic controller 114 may be configured to send data received from the user interface 110 to the software app 208 via the communication interface 112 for processing on the phone 206.

In one embodiment, the storage or memory device 116 includes any physical hardware storage or memory device capable of storing data in any format, including encrypted data. Various types of data described herein may be stored on the storage or memory device 116 during authentication operations and before or after such operations. In some embodiments, data stored on the storage or memory device 116 is locked with access controls that are only available to specific users or components of the system 200. For example, some of the data stored on the storage or memory device 116 may be available for write operations by only the app 208 in response to instructions from the authentication system 204.

In one embodiment, the power source 118 includes any type of power source and/or drivers useful in the operation of the battery 102. The power source 118 may be used to power any or all of the other components within the battery 102. Additionally, the power source 118 may be connected via power terminals (not shown) to the pod 104 for supplying adequate power to the pod for its internal operations.

FIG. 4 depicts a schematic diagram of one embodiment of the pod 104 of FIG. 1. The illustrated pod 104 includes a dispenser module 120, a communication interface 122, and a physical code 124. Although the pod 104 is shown and described with certain components and functionality, other embodiments of the pod 104 may include fewer or more components to implement similar or different functionality.

In one embodiment, the dispenser module 120 is configured to securely contain medication or another substance prior to authorized access by an authorized user. The dispenser module 120 may include various physical features related to dispensing the medication or other substance. For example, in some embodiments the dispenser module 120 includes a heater element (not shown) that is powered to a temperature sufficient to vaporize some or all of the medication or other substance stored in the dispenser module 120. Additionally, the dispenser module 120 may include any ports and/or valves necessary for the physical dispensing of the medication or other substance out of the dispenser module 120 to the user.

In one embodiment, the communication interface 122 facilitates electronic or digital communications directly with communication interface 112 of the battery 102. In other embodiments, the communication interface 122 also facilitates electronic or digital communications, either directly or indirectly, with other components of the system 200. Similar to the communication interface 112, the communication interface 122 includes hardware and controllers to facilitate communications using any available communication protocol such as Wi-Fi, Bluetooth, near field communication (NFC), or other communication protocols, as well as any standardized or proprietary communication protocol over direct hardwired contacts with the battery 102.

In one embodiment, the physical code 124 is any type of identifier or code affixed to or applied to the pod. The physical code 124 may be visually apparent to a user or, alternatively, may be hidden or detectable only by electronic or other non-visual hardware. As one example, the physical code 124 may be a QR code attached as a sticker to an outside surface of the pod 104, in which case the QR code may carry or encrypt specific data about the medication in the dispenser module 120, the user authorized for use of the pod 104, or the dosage specifications for the user. In other embodiments, other types of information may be stored in connection with the physical code 124. Other types of physical code formats may be used for the physical code 124.

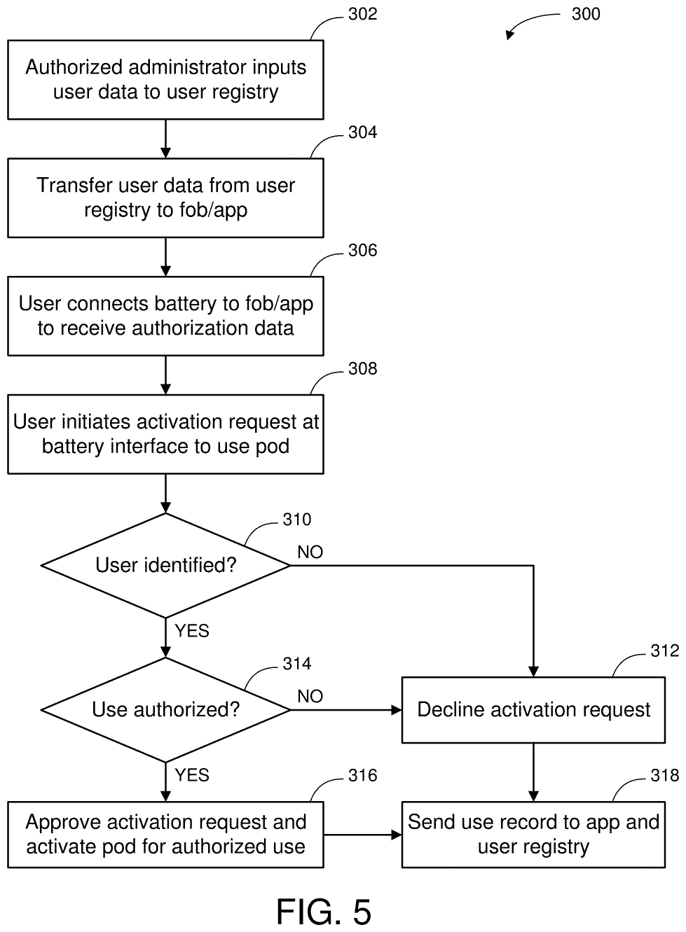

FIG. 5 depicts a flow chart diagram of one embodiment of a method 300 for authenticating user requests. Although the method 300 is described in conjunction with the vaporization apparatus 100 and components of the system 200 of FIGS. 1 and 2, embodiments of the method 300 may be implemented with other types of vaporization or dispenser apparatuses and/or authorization systems.

In the depicted embodiment, the method 300 begins when an authorized administrator inputs 302 data about the user into a user registry within the authorization system 204. The administrator, or another agent within the system 200, then transfers 304 that user data from the user registry to the fob 210 and/or the application 208. If stored on the fob 210, the fob may be delivered to the end user. The user then connects 306 the battery 102 to the fob 210 or application 208 to receive some or all of the user data from the fob 210 or application 208.

Subsequently, when a user initiates 308 an activation request at the battery 102 to use the pod 104 for dispensing medication or another substance, the pod 104 and battery 102 attempt to authenticate the user for the requested dispensing of the medication. The specific type of authentication that occurs may vary depending on the user making their request, the setup established by the authorization system 204, the qualification criteria required by the administrator, or a number of other factors capable of customization and validation relative to a specific user or a specific type of medication or other substance.

In the illustrated embodiment, the electronic controller 114 within the battery 102 attempts to identify 310 the user. This may occur, for example, by an input into the user interface 110 that is unique to a specific user. For example, a user may input a fingerprint, a voice command, or another input code unique to that user. If the electronic controller 114 is unable to identify the user who is authorized to use the corresponding pod 104, then the electronic controller 114 declines 312 the activation request by the user.

Otherwise, if the electronic controller 114 is able to identify 310 the correct user, then the electronic controller 114 proceeds to confirm 314 if the requested use is authorized. Any number or combination of constraints may be placed on a user's authorization to access and dispense a particular medication or substance from the pod 114. For example, a user may be subjected to time constraints on when the pod 104 may dispense medication or another substance. More specifically, electronic controller 114 may limit the frequency between dosages dispensed to the user. Alternatively, or additionally, the electronic controller 114 may limit the amount of specific dosages or the concentration of specific dosages for any given dosage period. Other embodiments may impose other constraints for specific uses authorized to a specific user.

If the electronic controller 114 determines that the user is correctly identified 310 and is authorized 314 to use the vaporization apparatus 100 at the requested time, then the electronic controller 114 approves 316 the activation request and activates the pod 114 for the authorized use. Whether the requested use is declined 312 or approved 316, the electronic controller 114 sends 318 a use record, or request log record, to the app 208 for storage in the user registry of the authorization system 204. The depicted method 300 then ends.

In further embodiments, an administrator may set up additional access to the medication or other substance in the pod 104 on a one time or exceptional basis. For example, if the correct user is identified by the electronic controller 114, then the user may have access to a supplemental dosage of medication outside of the normal dosage constraints set by the administrator.

Although the operations of the method(s) herein are shown and described in a particular order, the order of the operations of each method may be altered so that certain operation may be performed in an inverse order or so that certain operations may be performed, at least in part, concurrently with other operations. In another embodiment, instructions or sub-operations of distinct operations may be implemented in an intermittent and/or alternating manner.

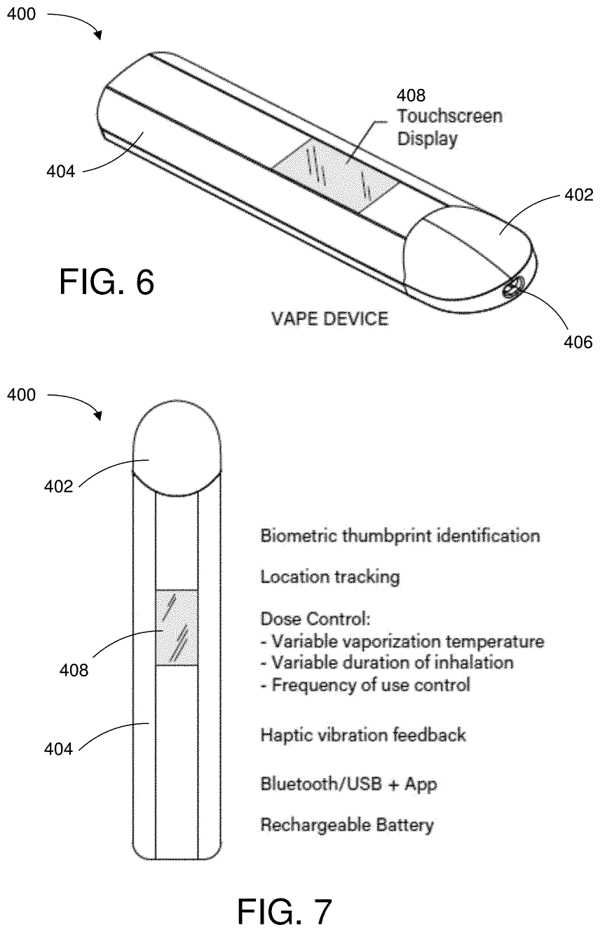

FIG. 6 depicts a schematic perspective diagram of one embodiment of a vape device 400. In the illustrated embodiment, the vape device 400 includes a mouthpiece 402 and a battery or control unit 404. The mouthpiece 402 inserts into an end of the battery 404, which provides power, as needed, to the mouthpiece 402. An orifice 406 in the mouthpiece 402 provides a channel for vaporized substances to exit the mouthpiece 402 for inhalation by a user.

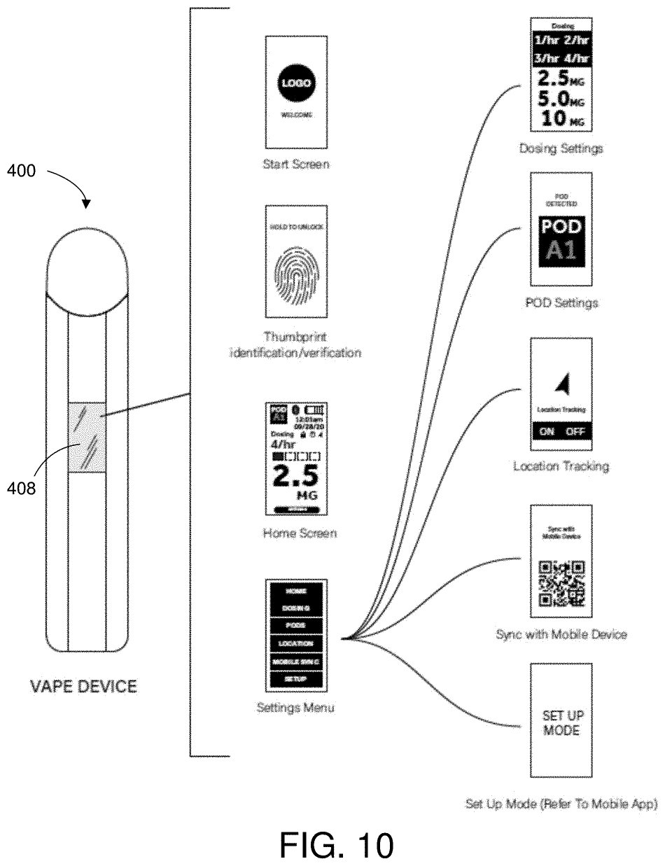

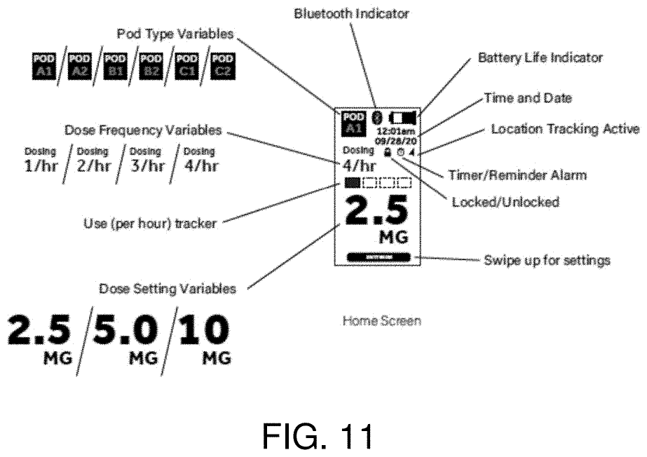

The illustrated embodiment of the vape device 400 also includes a touch screen display 408. The touch screen display 408 may be implemented on any surface or surfaces of the battery 404. Additionally, the touch screen display 408 may include one or more discreet graphical user interfaces or touch interfaces. Several examples of data that maybe communicated by the battery or control unit 404 to a user are shown in FIGS. 10 and 11 and described in more detail below. Additionally, the touch screen display 408 may be configured to accept user inputs in any manner compatible with the technology of the touch screen display 408.

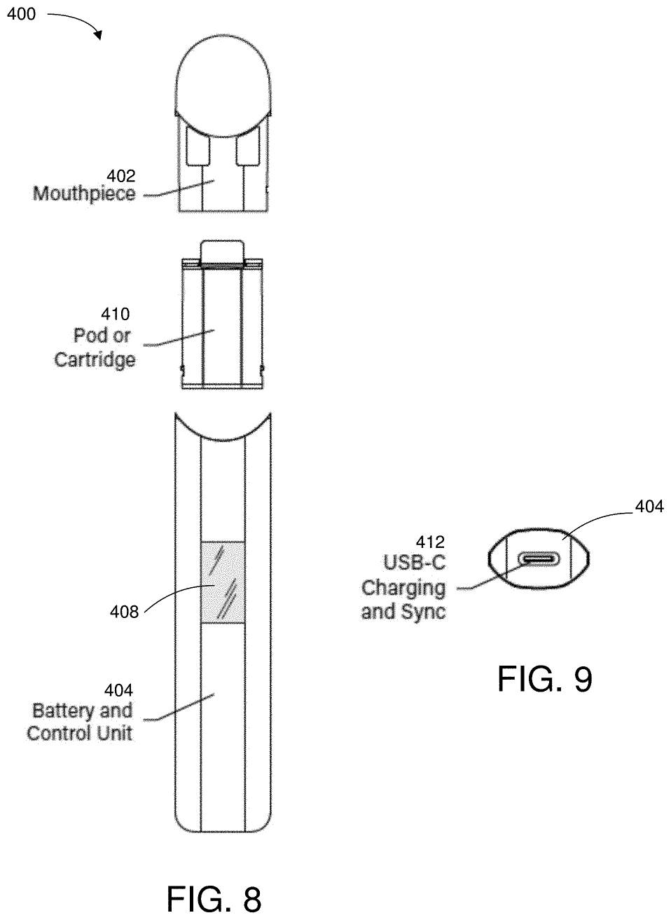

FIG. 7 depicts a plan view of the vape device of FIG. 6. FIG. 7 also lists examples of functions that may be implemented by the vape device 400 using the touchscreen display 408 or any other interface of the battery 404 or the mouthpiece 402. As examples of the types of functions that may be implemented, FIG. 7 lists biometric thumbprint identification, location tracking of the battery 404 or the mouthpiece 402 or a pod 410 as shown in FIG. 8, dose control through various combinations of control criteria (such as variable vaporization temperature, variable duration of inhalation, and frequency of use control), and haptic vibration feedback. Additionally, the battery or control unit 404 may provide Bluetooth, USB, or other communication protocol functionality to interface with an app on a phone or another remote device. Also, in some embodiments, the battery 404 is a rechargeable battery.

FIG. 8 depicts an exploded view of the vape device 400 of FIG. 6. The illustrated vape device 400 includes a mouthpiece 402 and a battery and control unit 404, as well as a pod or cartridge 410. The pod 410 includes a vaporization substance or medical substance that may be administered through the mouthpiece 402 to a user under the control of the battery and control unit 404. Various types of direct, physical, or wireless power and communications may be transmitted among the mouthpiece 402, the battery in control unit 404, and the pod 410.

FIG. 9 depicts one embodiment of an end view of the vape device 400 of FIG. 6. The battery and control unit 404, in this embodiment, includes a charging port 412 to receive power from an external power source (not shown). The port 412 also might be used for synchronizing certain data communications with another device. In some embodiments the port is a USB-C compatible port, although other physical types of port interconnects may be used with other communication protocols.

FIG. 10 depicts one embodiment of various interface messages of the vape device 400 of FIG. 6. Although various messages are shown and described herein, other embodiments may implement additional or different messages, or different content for similar messages, within the scope of the embodiments herein.

Depicted examples of content for the touch screen display 408 include a start screen, a thumbprint identification and verification screen, a home screen, and a settings menu. Each of these screens may facilitate entry into one or more other screens within the functionality of the control unit 404. For example, the settings menu may allow a user to access various settings including, but not limited to, dosing settings, pod settings, location tracking, synchronization settings with a mobile device, and a setup mode.

FIG. 11 depicts embodiments of interface messages of the vape device 400 of FIG. 6. The illustrated messages show examples of the types of information and communications that may be provided on the home screen using the touch screen display 408. As examples, although not limiting, the home screen may include pod type indicators to show the type of pod or content of the pod in use with the mouthpiece 402, dose frequency variables such as time intervals between allowable doses, usage tracking, dose setting variables for volume or percentage amounts of dosages, and so forth. Additional information or indicators may include, but are not limited to, a Bluetooth indicator, a battery life indicator, time and date indicators, location tracking activity indicators, a timer or alarm, a lock status indicator, and verbal or graphical instructions for user interface with the touch screen display 408. Other embodiments may include additional or different information, communications, or indicators.

Although the functions and components described herein refer primarily to vaporization types of substances, other embodiments of the systems and devices within the scope of this description may include various types of substances for delivery to a user. For example, in some embodiments, the substance or medication delivered to a user may be in a pill format. In other embodiments, the substance or medication delivered to a user may be in a liquid format. The type of substance or medication delivered to a user does not necessarily limit the scope of this description in any way.

In the above description, specific details of various embodiments are provided. However, some embodiments may be practiced with less than all of these specific details. In other instances, certain methods, procedures, components, structures, and/or functions are described in no more detail than to enable the various embodiments of the invention, for the sake of brevity and clarity.

Although specific embodiments of the invention have been described and illustrated, the invention is not to be limited to the specific forms or arrangements of parts so described and illustrated. The scope of the invention is to be defined by the claims appended hereto and their equivalents.

Furthermore, embodiments of the invention can take the form of a computer program product accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device.

The computer-useable or computer-readable medium can be an electronic, magnetic, optical, electromagnetic, infrared, or semiconductor system (or apparatus or device), or a propagation medium. Examples of a computer-readable medium include a semiconductor or solid-state memory, magnetic tape, a removable computer diskette, a random-access memory (RAM), a read-only memory (ROM), a rigid magnetic disk, and an optical disk. Current examples of optical disks include a compact disk with read only memory (CD-ROM), a compact disk with read/write (CD-R/W), and a digital video disk (DVD).

Input/output or I/O devices (including but not limited to keyboards, displays, pointing devices, etc.) can be coupled to the system either directly or through intervening I/O controllers. Additionally, network adapters also may be coupled to the system to enable the data processing system to become coupled to other data processing systems or remote printers or storage devices through intervening private or public networks. Modems, cable modems, and Ethernet cards are just a few of the currently available types of network adapters.

What is claimed is:

1. A battery for a vaporization apparatus, the battery comprising:
a data storage device configured to read authorization data for an authorized user of the vaporization apparatus;
a user interface configured to receive a security input from a user; and
an electronic controller configured to communicate electronically with the user interface and the data storage device,
wherein the electronic controller is further configured to receive the security input from the user interface, compare the received security input with the authorization data to identify the authorized user in an authentication process, and authorize use of the vaporization apparatus.

2. The battery of claim 1, wherein the electronic controller is configured to communicate with an authentication system wherein the authentication system is configured to communicate with the data storage device.

3. The battery of claim 2, wherein the electronic controller executes an authentication process to generate an identification determination of the authorized user.

4. The battery of claim 3, wherein the electronic controller is configured to receive the input from the user interface and compare the received input with the authorization data, wherein the authorization data is stored in the data storage device.

5. The battery of claim 2, wherein the authentication system executes an authentication process to generate an identification determination of the authorized user.

6. The battery of claim 5, wherein the authentication system is configured to receive the input from the user interface and compare the received input with the authorization data stored in the data storage device to generate the identification determination of the authorized user.

7. The battery of claim 1, wherein the battery activates a pod upon authorized activation of the battery by the electronic controller.

8. The battery of claim 7, wherein the pod comprises a heating element configured to heat up the vaporized substance upon activation of the pod.

9. The battery of claim 1, wherein the user interface is further configured to receive a request to use the vaporization apparatus from the user.

10. The battery of claim 9, wherein the electronic controller is configured to authorize the request from the user in response to an identification determination and further authorize a specific dosage of vaporized substance.

11. A smart vaporization device, the smart vaporization device comprising:
a pod comprising a heating element and smart dispenser module, wherein the smart dispenser module is configured to contain and dispense an authorized dosage of a vaporizable substance to an authorized user of the smart vaporization device; and
a battery connected to the pod, wherein the battery comprises:
a data storage device configured to store authorization data about the authorized user;
an electronic controller connected to the heating element of the pod, wherein the electronic controller is in communication with the data storage device, wherein the electronic controller is configured to activate the heating element of the pod in response to an identification determination of the authorized user; and
a user interface configured to receive a user input from a user, wherein the user interface is in electronic communication with the electronic controller.

12. The smart vaporization device of claim 11, wherein the electronic controller is configured to generate the identification determination of the authorized user.

13. The smart vaporization device of claim 12, wherein the electronic controller is configured to receive the input from the user interface in generating the identification determination of the authorized user.

14. The smart vaporization device of claim 11, wherein the electronic controller is configured to be in communication with an authentication system.

15. The smart vaporization device of claim 14, wherein the authentication system is configured to generate the identification determination of the authorized user.

16. The smart vaporization device of claim 11, wherein an authorized administrator uploads the authorization data about the authorized user to the data storage device.

17. The smart vaporization device of claim 11, wherein the authorized dosage is controlled by a temperature and an amount of time of heat applied to the smart dispenser module by the heating element.

18. A system for dispenser authorization controls comprising:
an authentication system comprising a data registry configured to store a user profile;
a vaporization apparatus comprising:
a pod having a smart dispenser module configured to dispense an authorized dosage to an authorized user; and
a battery having an electronic controller, wherein the electronic controller is in communication with the authentication system,
wherein the electronic controller is further configured to activate the pod based upon identification of the authorized user; and
a device in communication with the authentication system, wherein the device is programmed with the user profile and is configured to set up the vaporization apparatus for dispenser authorization controls.

19. The system of claim 18, wherein the electronic controller is configured to access the user profile in the data registry of the authentication system in generating the identification of the authorized user.

20. The system of claim 18, wherein the electronic controller is configured to send a use record of the vaporization apparatus to the authentication system and further control an access frequency to the vaporization apparatus.
